(19) United States
(12) Patent Application Publication
(10) Pub. No.: US 2025/0019622 A1
(43) Pub. Date: Jan. 16, 2025

(54) CLEANING AGENT

(71) Applicant: SHISEIDO COMPANY, LTD., Tokyo (JP)

(72) Inventors: Takashi MENO, Tokyo (JP); Chihiro MASUSAWA, Tokyo (JP)

(73) Assignee: SHISEIDO COMPANY, LTD., Tokyo (JP)

(21) Appl. No.: 18/713,357

(22) PCT Filed: Dec. 14, 2022
(86) PCT No.: PCT/JP2022/045964

§ 371 Date: May 24, 2024

(30) Foreign Application Priority Data
Dec. 27, 2021 (JP) 2021-213048

(51) Int. Cl. C11D 7/14; C11D 7/12; C11D 7/20

(52) U.S. Cl. CPC C11D 7/14; C11D 7/12; C11D 7/20

(57) ABSTRACT

The present invention provides a cleaning agent which is free from contamination of wastewater and is excellent in cleaning property and sensory experience. A cleaning agent according to the present invention includes: (A) a clay mineral; and (B) one or two or more inorganic salts selected from the group consisting of sodium carbonate, sodium bicarbonate, lithium carbonate, and lithium bicarbonate. In this cleaning agent, the total content of an organic substance based on the total mass of the cleaning agent is 2% by mass or less.

### TECHNICAL FIELD

The present invention relates to a cleaning agent containing no organic carbon and having a high cleaning effect.

### BACKGROUND ART

Recently, development of environmentally friendly cleaning agents has been desired. This is because wastewater after use of a synthetic cleaning agent or the like leads to environmental pollution, and therefore it is desirable to prevent such pollution. Such cleaning agents have been studied not only for clothing or dishware/food cleaning agents but also for body soaps, shampoos, facial cleansers, and the like.

However, many of them are not synthetic materials but natural materials. Such cleaning agents have a lower environmental burden than cleaning agents using synthetic materials. However, since even a natural component contains an organic substance, the wastewater after use thereof contains an organic substance. As a result, it cannot be said that the nutritional value in the wastewater is low, and bacteria may propagate. The degree of contamination of the wastewater is evaluated by a biochemical oxygen demand (BOD) and a chemical oxygen demand (COD), but when bacteria propagate in the wastewater, BOD and COD increase. Therefore, in order to suppress an increase in BOD and COD in the wastewater, it is necessary to reduce the content of the organic material in the wastewater. However, smear removal with body soaps and the like is generally achieved by an organic material such as a surfactant, and it has been difficult to achieve a cleaning effect without using an organic material.

On the other hand, as an environmentally friendly cleaning agent, there is known a cosmetic composition characterized by using a solution in which crystals containing negatively hydrated ions proposed by Samoilov are dissolved (Patent Literature 1). This literature reveals that, in order to lower an organic substance content so as to solve the problem of the safety inhibition by an organic substance, a sufficient effect can be obtained by using a salt containing a negative hydrated ion, that is, an ion such as a potassium ion, a rubidium ion, or an ammonium ion, but an effect obtained by a potassium ion or the like cannot be obtained by a sodium ion or the like. The literature also describes the use of an organic substance such as EDTA for this cosmetic, and it is insufficient as a composition containing no organic substance.

### CITATION LIST

### Patent Literature

Patent Literature 1: JP 2000-143489 A

### SUMMARY OF THE INVENTION

In view of the above problems, an object of the present invention is to provide a cleaning agent that achieves sufficient cleanability and sensory experience even though the content of organic carbon is extremely low.

According to the present invention, the following invention is provided.
[1] A cleaning agent including:
   (A) a clay mineral; and
   (B) one or two or more inorganic salts selected from the group consisting of sodium carbonate, sodium bicarbonate, lithium carbonate, and lithium bicarbonate,
   wherein a total content of an organic substance is 2% by mass or less based on a total mass of the cleaning agent.
[2] The cleaning agent according to [1], in which the component (A) is a layered silicate mineral.
[3] The cleaning agent according to [1] or [2], in which the component (A) is smectite.
[4] The cleaning agent according to any one of [1] to [3], in which the component (A) is selected from the group consisting of montmorillonite, beidellite, saponite, stevensite, and hectorite.
[5] The cleaning agent according to any one of [1] to [4], further including an inorganic powder (C) other than the component (A) or the component (B).
[6] The cleaning agent according to any one of [1] to [5], in which the component (C) is selected from the group consisting of silica, zeolite, alumina, titanium oxide, zeolite, mica, and talc.
[7] The cleaning agent according to any one of [1] to [6], in which a weight reduction rate when the cleaning agent is dried at 105° C. for 2 hours is 9% or less.
[8] The cleaning agent according to any one of [1] to [7], in which the cleaning agent does not contain an organic substance or water.
[9] The cleaning agent according to any one of [1] to [8], in which the content of the component (A) is 10 to 90% by mass and the content of the component (B) is 2 to 60% by mass based on the total mass of the cleaning agent.
[10] The cleaning agent according to any one of [1] to [9], in which a ratio B/A of the content of the component (B) to the content of the component (A) is 0.05 to 0.3.
[11] The cleaning agent according to any one of [1] to [10], in which the cleaning agent has a viscosity of 300 to 400,000 Pas at 20° C. when water, the amount of which is 9 times the mass of the cleaning agent, is added to the cleaning agent and uniformly mixed.
[12] The cleaning agent according to any one of [1] to [11], in which the cleaning agent has a pH of 7 to 9 at 20° C. when water, the amount of which is 9 times the mass of the cleaning agent, is added to the cleaning agent and uniformly mixed.

According to the present invention, there is provided a cleaning agent capable of achieving sufficient cleanability and sensory experience while substantially eliminating contamination of wastewater, specifically, an increase in BOD, COD, or COD.

### SPECIFIC DESCRIPTION OF THE INVENTION

A cleaning agent according to the present invention includes:
(A) a clay mineral; and
(B) one or two or more inorganic salts selected from the group consisting of sodium carbonate, sodium bicarbonate, lithium carbonate, and lithium bicarbonate. Furthermore, the total content of an organic substance with respect to the total mass of the cleaning agent is 2% by mass or less.

In the present invention, an “organic substance” means a compound containing carbon, but does not include a simple compound containing carbon and a carbon allotrope. Here, the simple compound containing carbon is, for example, carbon monoxide, carbon dioxide, a metal carbonate, a metal bicarbonate, or the like, and the carbon allotrope is graphite, diamond, or a carbon cluster (fullerene or the like). On the other hand, an “inorganic substance” is a substance other than the organic substance.

In the cleaning agent according to the present invention, the content of the organic substance is 2% by mass or less, preferably 1% by mass or less, and most preferably does not contain water, based on the total mass of the cleaning agent. When water is contained, generation of bacteria is likely to occur at the time of storage, and a preservative (that is, an organic substance) is also required in order to prevent the generation of bacteria, but since the cleaning agent according to the present invention is used by blending water only at the time of use, no bacteria are generated at the time of storage, and the time from blending of water to discharge is short, there is no time for bacteria to propagate, so that the wastewater is not contaminated.

The cleaning agent according to the present invention includes a clay mineral (hereinafter, may be referred to as component (A)). In the present invention, a clay mineral (A) is generally composed mainly of fine layered silicate minerals contained in natural minerals, and can be selected as desired from those applied to applications such as cosmetics. Among such clay minerals, high purity clay from which impurities have been removed or synthetic clay minerals can also be used.

Clay minerals classified as layered silicate minerals are further classified as 1:1 type minerals such as kaolinite, 2:1 type minerals such as smectite, 2:1:1 minerals such as chlorite, and among these, smectite is preferably used. Furthermore, among smectites, those selected from the group consisting of montmorillonite, beidellite, saponite, stevensite, and hectorite are preferably used.

Specifically, a group of natural or synthetic montmorillonites (VEEGUM (trade name, manufactured by Vanderbilt Minerals, LLC), KUNIPIA (trade name, manufactured by KUNIMINE INDUSTRIES CO., LTD.), LAPONITE (trade name, manufactured by BYK Japan KK), etc.) such as montmorillonite, saponite, and hectorite can be used. In addition, synthetic saponite in which a part of magnesium of saponite is substituted with aluminum can also be known. Incidentally, modified clay minerals obtained by modifying clay minerals with an organic material are also known, but since these contain organic carbon, it is preferable not to use them.

The content of the clay mineral (component (A)) can be selected as desired according to the purpose, but is preferably 10 to 90% by mass and more preferably 30 to 60% by mass based on the total mass of the cleaning agent in order to realize sufficient cleaning property.

In addition, the cleaning agent according to the present invention contains an inorganic salt (hereinafter, may be referred to as component (B)). This inorganic salt is selected from the group consisting of sodium carbonate, sodium bicarbonate, lithium carbonate, and lithium bicarbonate, and can be used alone or in combination of two or more thereof. In the present invention, it is preferable that even an inorganic salt does not contain other water-soluble inorganic salts.

The content of the inorganic salt (component (B)) can be selected as desired according to the purpose, but is preferably 2 to 60% by mass and more preferably 5 to 20% by mass based on the total mass of the cleaning agent in order to realize sufficient cleaning property.

In addition, the cleaning property and the sensory experience can be adjusted by the blending ratio of the component (A) and the component (B). In order to achieve both the cleaning property and the sensory experience, the ratio B/A of the content of the component (B) to the content of the component (A) is preferably 0.05 to 0.3.

The cleaning agent according to the present invention can further contain another inorganic powder (hereinafter, may be referred to as a component (C)) in addition to the components (A) and (B). By using such an inorganic powder, the cleaning property can be further improved.

It is considered that such an inorganic powder realizes a so-called scrubbing effect to improve the cleaning property. The inorganic powder can be selected as desired from those generally used in the fields of cosmetics and pharmaceuticals, and for example, those selected from the group consisting of silica, zeolite, alumina, titanium oxide, zeolite, mica, and talc are preferable. The particle diameter of these inorganic powders is not particularly limited, but is preferably 1 mm or less, and more preferably 700 μm or less, in order to improve the sensory experience at the time of washing. The particle shape is preferably close to a true sphere. Incidentally, inorganic particles whose surfaces have been subjected to a hydrophobic treatment or a hydrophilic treatment are also known, but inorganic particles using an organic material in the treatment should not be used for the cleaning agent according to the present invention.

The cleaning agent according to the present invention contains the component (A) and the component (B), and contains the component (C) if necessary. If necessary, an inorganic substance can be blended as another component, but an organic substance is not blended. In addition, since water may promote the propagation of bacteria, it is preferable not to blend water. When the cleaning agent according to the present invention is stored in a dry state without water, propagation of fungi does not occur. Therefore, even if water is blended at the time of use, BOD and COD of wastewater do not increase. Therefore, the moisture content of the cleaning agent according to the present invention is preferably low. Specifically, the weight reduction rate when the cleaning agent is dried at 105° C. for 2 hours is preferably 9% or less, and more preferably 7%.

The cleaning agent according to the present invention is generally stored in a dry state and are diluted with water at the time of use for cleaning. The dilution rate at the time of use is not limited, but is generally 2 to 20 times, and preferably 5 to 15 times. At this time, the viscosity and pH vary particularly depending on the type of clay mineral. Here, from the viewpoint of ease of use at the time of use, it is preferable that the viscosity when diluted with water is in a specific range. Specifically, when water, the amount of which is 9 times the mass of the cleaning agent, is added to the cleaning agent and uniformly mixed, the viscosity at 20° C. is preferably 300 to 400,000 Pa·s. Here, the viscosity is measured using a VDA type viscometer (DIGITAL VISMETRON VDA manufactured by Shibaura Systems Co., Ltd.) at 25° C. and a rotation speed of 10 rpm using an appropriate rotor.

In addition, when the pH at the time of dilution with water is too high or too low, excessive stimulation may be given to the skin. For this reason, when water, the amount of which is 9 times the mass of the cleaning agent, is added to the cleaning agent and uniformly mixed, the pH at 20° C. is preferably 7 to 9.

### EXAMPLES

The present invention will be described below using examples. Note that the present invention is not limited by these examples at all. In addition, the content is % by mass with respect to the total amount unless otherwise specified.

### Examples 1 to 4 and Comparative Examples 1 to 7

Components were blended so as to have the content ratios shown in Table 1 to prepare cleaning agents. The physical properties of the obtained cleaning agents were evaluated as follows.

### [Viscosity]

Each of the cleaning agents was diluted at the magnification shown in the table by adding water, and uniformly mixed, and the viscosity at 25° C. was measured. For the measurement, a VDA type viscometer (DIGITAL VISMETRON VDA manufactured by Shibaura Systems Co., Ltd.) was used.

[pH]

Water, the amount of which is 9 times the mass of the cleaning agent, was added to the cleaning agent and uniformly mixed, and the pH at 25° C. was measured.

The cleaning agent was further diluted at the ratio described in the table, and the properties of the composition was evaluated according to the following criteria.

### [Ease of Use]

The diluted cleaning agent was dispensed into a hand and evaluated by the state when inclined by 45°.
A: Does not drip
B: Takes time to drip
C: Immediately drip

### [Skin Cleaning Property]

The diluted cleaning agent was applied to the inside of an arm over which squalane has been spread, and rubbed 20 times with a finger and then washed away. After washing away, the skin cleaning property was evaluated by feeling and appearance observation (gloss level) of squalane remaining on the arm.
A: Remaining squalane is not completely observed
B: Remaining squalane is hardly observed
C: Remaining squalane is observed

### [Table 1]

TABLE 1

| | | Compar- | | | | | | | Compar- | Compar- |
|---|---|---|---|---|---|---|---|---|---|---|
| | | ative | | | | | | | ative | ative |
| Component | | example 1 | Example 1 | Example 2 | Example 3 | Example 4 | Example 5 | Example 6 | example 2 | example 3 |
| A | Bentonite*¹ | | 60 | 60 | 60 | | | | | 70 |
| | Bentonite*² | | | | | 90 | 90 | | | |
| | Silicic acid (Al/Mg) | | | | | | | 90 | 100 | |
| B | Sodium bicarbonate | 100 | 10 | 10 | 10 | 10 | 10 | 10 | | |
| C | Silica | | 30 | 30 | 30 | | | | | 30 |
| Physical | Viscosity | — | 207000 | 1511 | 437 | 189000 | 82000 | 383000 | 483000 | 2500 |
| property | pH | 8.14 | 8.29 | 7.78 | 7.72 | 8.46 | 8.98 | 8.78 | 9.15 | 7.23 |
| Evaluation | Dilution rate | 100 times | 5 times | 10 times | 15 times | 10 times | 15 times | 10 times | 10 times | 10 times |
| | Ease of use | C | A | A | B | A | A | A | A | A |
| | Sebum cleaning property | B | A | A | A | A | A | A | C | B |

*¹KUNIPIA G (manufactured by KUNIMINE INDUSTRIES CO., LTD.)
*²KUNIPIA-G4 (manufactured by KUNIMINE INDUSTRIES CO., LTD.)

### [Measurement of Total Organic Carbon and Biochemical Oxygen Demand of Cleaning Wastewater]

For the cleaning agent of Example 2, the total organic carbon (TOC) and the biochemical oxygen demand (BOD) when it was used were measured.

A face wash of a human was performed using 1 g (specified amount) of the cleaning agent, and then rinsed with rinsing water to collect the rinsing water after use.

The collected rinsing water was stirred so as to be uniform, and then the TOC was measured according to JIS K 0102 22.1 0.5 combustion oxide-infrared TOC analysis method (in terms of C), and the BOD was measured according to JIS K 0102 21 and 32.3 diaphragm electrode method.

Here, the amount of rinsing water after use was 4.5 L as a standard amount. When the amount of the collected rinsing water was less than 4.5 L, water was added up to 4.5 L, and then the measurement was performed. When the amount of the collected rinsing water exceeded 4.5 L, the collected rinsing water was measured and corrected by the following equation.

Corrected TOC=(amount of rinsing water collected [L]/4.5 [L])×measured TOC

Corrected BOD=(amount of rinsing water collected [L]/4.5 [L])×measured BOD

This operation was performed by 12 persons, and TOC and BOD of the collected rinsing water were measured for each of the operations, and an average value thereof was determined.

The obtained average TOC was 4 mg/L, and the obtained average BOD was 4 mg/L. The daily average of the National Effluent Standards defined by the Ministry of the Environment is BOD 120 mg/L, and the contamination of the environment with organic substances is very slight when the cleaning agent according to the present invention is used.

1. A cleaning agent comprising:
(A) a clay mineral; and
(B) one or two or more inorganic salts selected from the group consisting of sodium carbonate, sodium bicarbonate, lithium carbonate, and lithium bicarbonate,
wherein a total content of an organic substance is 2% by mass or less based on a total mass of the cleaning agent.

2. The cleaning agent according to claim 1, wherein the component (A) is a layered silicate mineral.

3. The cleaning agent according to claim 1, wherein the component (A) is smectite.

4. The cleaning agent according to claim 1, wherein the component (A) is selected from the group consisting of montmorillonite, beidellite, saponite, stevensite, and hectorite.

5. The cleaning agent according to claim 1, further comprising an inorganic powder (C) other than the component (A) or the component (B).

6. The cleaning agent according to claim 1, wherein a component (C) is selected from the group consisting of silica, zeolite, alumina, titanium oxide, zeolite, mica, and talc.

7. The cleaning agent according to claim 1, wherein a weight reduction rate when the cleaning agent is dried at 105° C. for 2 hours is 9% or less.

8. The cleaning agent according to claim 1, wherein the cleaning agent does not contain an organic substance or water.

9. The cleaning agent according to claim 1, wherein a content of the component (A) is 10 to 90% by mass and a content of the component (B) is 2 to 60% by mass based on a total mass of the cleaning agent.

10. The cleaning agent according to claim 1, wherein a ratio B/A of a content of the component (B) to a content of the component (A) is 0.05 to 0.3.

11. The cleaning agent according to claim 1, wherein the cleaning agent has a viscosity of 300 to 400,000 Pa's at 20° C. when water, an amount of which is 9 times a mass of the cleaning agent, is added to the cleaning agent and uniformly mixed.

12. The cleaning agent according to claim 1, wherein the cleaning agent has a pH of 7 to 9 at 20° C. when water, an amount of which is 9 times a mass of the cleaning agent, is added to the cleaning agent and uniformly mixed.
